(19) United States
(12) Patent Application Publication
(10) Pub. No.: US 2020/0243177 A1
(43) Pub. Date: Jul. 30, 2020

(54) MEDICAL REPORT GENERATING DEVICE AND MEDICAL REPORT GENERATING METHOD

(71) Applicant: CANON MEDICAL SYSTEMS CORPORATION, Otawara-shi (JP)

(72) Inventors: Koichi TERAI, Shioyagun (JP); Hiroki SAITO, Otawara (JP); Hiroyoshi CHIYO, Nasushiobara (JP); Hirobumi NONAKA, Yaita (JP); Takayuki TANEMOTO, Otawara (JP)

(73) Assignee: CANON MEDICAL SYSTEMS CORPORATION, Otawara-shi (JP)

(21) Appl. No.: 16/775,385

(22) Filed: Jan. 29, 2020

(30) Foreign Application Priority Data
Jan. 30, 2019 (JP) 2019-014209

(51) Int. Cl. G16H 15/00; G16H 30/20; G16H 10/60

(52) U.S. Cl. CPC G16H 15/00; G16H 10/60; G16H 30/20

(57) ABSTRACT

A medical report generating device according to an embodiment includes processing circuitry. The processing circuitry is configured to obtain a medical report generated in the past. The processing circuitry is configured to generate a first medical report serving as a current medical report on the basis of the past medical report and to identify at least one update candidate item that needs to be updated in the first medical report. The processing circuitry is configured to cause a display to display the identified update candidate item in a recognizable manner.

### CROSS-REFERENCE TO RELATED APPLICATIONS

This application is based upon and claims the benefit of priority from Japanese Patent Application No. 2019-014209, filed on Jan. 30, 2019; the entire contents of which are incorporated herein by reference.

### FIELD

Embodiments described herein relate generally to a medical report generating device and a medical report generating method.

### BACKGROUND

In medical facilities such as hospitals, medical examinations are regularly performed on patients to observe progresses of conditions of diseases. Image interpreting doctors perform image interpretation by using medical image data or the like acquired in the medical examinations and generates image interpretation reports (medical reports). For example, an image interpreting doctor may generate a medical report (a medical report at this time; hereinafter, “current medical report”) when having performed a medical examination on a patient at this time for the purpose of observing the progress. In this situation, when generating the current medical report, the image interpreting doctor may make a copy of a medical report (a past medical report) that was generated at the time of a medical examination performed on the patient in the past.

When using the copy of the past medical report, the image interpreting doctor generates the current medical report by updating a part of the past medical report that was copied. In that situation, when generating the current medical report, the image interpreting doctor needs to determine for himself/herself the items that need to be updated (hereinafter, “update candidate items”), while ensuring that none of the update candidate items are overlooked.

### BRIEF DESCRIPTION OF THE DRAWINGS

FIG. 1 is a diagram illustrating an exemplary configuration of a medical report generating system including a medical report generating device according to an embodiment;

FIG. 2 is a diagram illustrating an exemplary configuration of the medical report generating device according to the present embodiment;

FIG. 3 is a flowchart illustrating a procedure in processes performed by the medical report generating device according to the present embodiment;

FIG. 4 is a drawing for explaining a process performed by the medical report generating device according to the present embodiment;

FIG. 5 is a drawing for explaining another process performed by the medical report generating device according to the present embodiment;

FIG. 6 is a drawing for explaining yet another process performed by the medical report generating device according to the present embodiment;

FIG. 7 is a drawing for explaining yet another process performed by the medical report generating device according to the present embodiment;

FIG. 8 is a flowchart illustrating a procedure in an update candidate item presenting process according to the present embodiment;

FIG. 9 is a drawing for explaining the update candidate item presenting process according to the present embodiment;

FIG. 10 is another drawing for explaining the update candidate item presenting process according to the present embodiment;

FIG. 11 is yet another drawing for explaining the update candidate item presenting process according to the present embodiment;

FIG. 12 is yet another drawing for explaining the update candidate item presenting process according to the present embodiment;

FIG. 13 is yet another drawing for explaining the update candidate item presenting process according to the present embodiment;

FIG. 14 is a flowchart illustrating a procedure in an update/confirmation process according to the present embodiment;

FIG. 15 is a drawing for explaining the update/confirmation process according to the present embodiment;

FIG. 16 is another drawing for explaining the update/confirmation process according to the present embodiment;

FIG. 17 is yet another drawing for explaining the update/confirmation process according to the present embodiment;

FIG. 18 is a flowchart illustrating a procedure in a saving process according to the present embodiment; and

FIG. 19 is a drawing for explaining the saving process according to the present embodiment.

### DETAILED DESCRIPTION

A medical report generating device according to an embodiment includes processing circuitry. The processing circuitry is configured to obtain a medical report generated in the past. The processing circuitry is configured to generate a first medical report serving as a current medical report on the basis of the past medical report and to identify at least one update candidate item that needs to be updated in the first medical report. The processing circuitry is configured to cause a display to display the identified update candidate item in a recognizable manner.

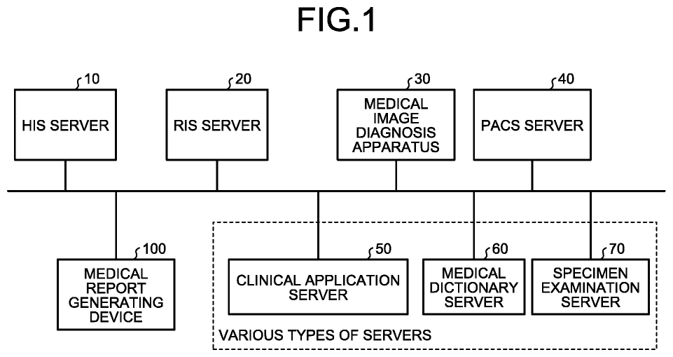

A medical report generating device that implements a medical report generating method according to an embodiment will be explained below, with reference to the accompanying drawings. In the following sections, an example of a medical report generating system including the medical report generating device will be explained. Although the medical report generating system illustrated in FIG. 1 includes one of each of the different types of apparatuses, the system in actuality may further include two or more apparatuses.

FIG. 1 is a diagram illustrating an exemplary configuration of the medical report generating system including a medical report generating device 100 according to the present embodiment. The medical report generating system illustrated in FIG. 1 includes: a Hospital Information System (HIS), a radiology department information management system (a Radiology Information System [RIS]), a medical image diagnosis apparatus 30, a medical image management system (a Picture Archiving and Communication System [PACS]), the medical report generating device 100, and various types of servers.

The HIS, the RIS, the medical image diagnosis apparatus 30, the PACS, the medical report generating device 100, and the various types of servers are connected to an intra-hospital Local Area Network (LAN) provided in a hospital, for example, and are each configured to transmit information to prescribed apparatuses and to receive information transmitted thereto from prescribed apparatuses. In addition to the connection to the intra-hospital LAN, the HIS may also be connected to an external network.

The HIS is a system configured to manage information occurring in the hospital. The HIS includes an HIS server 10. In the following sections, explanations will be omitted about terminals (HIS terminals) used by medical staff to perform various types of work in the hospital.

The HIS server 10 is configured to manage, in the HIS, the information occurring in the hospital. The information occurring in the hospital includes patient information, medical examination order information (hereinafter, simply “examination order information”), and the like. Records included in the patient information each have items such as a patient ID, the patient's name (the family name and the given name), the age (the date of birth), the gender, the height, the weight, the blood type, and the like. Records included in the examination order information each have items such as a medical examination ID (hereinafter, simply “examination ID”) identifying a medical examination (hereinafter, simply “examination”), a patient ID, information indicating whether the patient is hospitalized or an outpatient, an examination code, a medical department, a site subject to an examination (hereinafter, “examined site”), an examination type, an examination scheduled date/time, and the like.

The examination ID is issued at the time of inputting the examination order information and is an identifier used for uniquely identifying the examination order information within the one hospital, for example. The patient ID is issued for each patient and is, for example, an identifier used for uniquely identifying the patient within the one hospital. The examination code is an identifier defined in the one hospital, for example, and is used for uniquely identifying an examination. The medical department indicates a division of a specialty field of the diagnosis and treatment in medical services, for example. More specifically, examples of the medical department include internal medicine department, a surgical department, and the like. Examples of the examined site include the brain, the kidneys, the lungs, the liver, and the like. Examples of the examination type include an X-ray examination, a Computed Tomography (CT) examination, and a Magnetic Resonance Imaging (MRI) examination.

When examination order information has been input by an examination requesting doctor, for example, the HIS server 10 transmits the examination order information that was input and the patient information identified from the examination order information, to the RIS. Further, in that situation, the HIS server 10 transmits the patient information to the PACS.

The RIS is a system configured to manage examination appointment information related to radiological examination work. The RIS includes an RIS server 20. In the following sections, explanations will be omitted about terminals (RIS terminals) used by radiological technologists and the like, for example, to perform various types of work in the radiology department.

The RIS server 20 is configured to manage, in the RIS, information related to radiological examination work. For example, the RIS server 20 is configured to receive examination order information transmitted thereto from the HIS server 10, to accumulate received pieces of examination order information after appending various types of setting information thereto, and to manage the accumulated pieces of information as examination appointment information. More specifically, when the RIS server 20 has received the patient information and the examination order information transmitted thereto from the HIS server 10, the RIS server 20 is configured to generate the examination appointment information necessary for causing the medical image diagnosis apparatus 30 to operate, on the basis of the received patient information and examination order information. The examination appointment information contains information necessary for performing the examination such as an examination ID, a patient ID, an examined site, and an examination type, for example.

The RIS server 20 is configured to transmit the generated examination appointment information to the medical image diagnosis apparatus 30.

The medical image diagnosis apparatus 30 is an apparatus configured to perform the examination by imaging the patient, or the like. Examples of the medical image diagnosis apparatus 30 include an X-ray diagnosis apparatus, an X-ray Computed Tomography (CT) apparatus, a magnetic resonance imaging (MRI) apparatus, a nuclear medicine diagnosis apparatus, and an ultrasound diagnosis apparatus. The medical image diagnosis apparatus 30 may be referred to as a modality apparatus.

On the basis of the examination appointment information transmitted thereto from the RIS server 20, for example, the medical image diagnosis apparatus 30 performs the examination. Further, the medical image diagnosis apparatus 30 generates examination execution information indicating execution of the examination and transmits the generated examination execution information to the RIS server 20. In that situation, the RIS server 20 receives the examination execution information from the medical image diagnosis apparatus 30 and outputs the received examination execution information to the HIS server 10 and to the medical report generating device 100, as most recent examination execution information. The HIS server 10 receives the most recent examination execution information and manages the received examination execution information. The medical report generating device 100 receives the most recent examination execution information and generates a medical report (explained later) according to the received examination execution information. The examination execution information contains the examination appointment information (the examination ID, the patient ID, the examined site, and the examination type), as well as the date of the examination, and the like.

Further, during the execution of the examination, the medical image diagnosis apparatus 30 is configured to generate medical image data by imaging the examined subject (the patient). Examples of the medical image data include X-ray CT image data, X-ray image data, MRI image data, nuclear medicine image data, and ultrasound image data. The medical image diagnosis apparatus 30 converts the generated medical image data into, for example, a format compliant with a Digital Imaging and Communication in Medicine (DICOM) standard. In other words, the medical image diagnosis apparatus 30 is configured to generate the medical image data to which a DICOM tag is appended as additional information.

The additional information contains, for example, the patient ID, the examination ID, an apparatus ID, an image series ID, and the like and is standardized according to the DICOM standard. The apparatus ID is information identifying the medical image diagnosis apparatus 30. The image series ID is information identifying one session of imaging process performed by the medical image diagnosis apparatus 30 and may include, for example, the site of the examined subject (the patient) that was imaged, the image generation time, a slice thickness, slice positions, and the like. For example, by performing a CT examination or an MRI examination, it is possible to obtain tomographic images taken on a plurality of slice positions are obtained as the medical image data.

The medical image diagnosis apparatus 30 is configured to transmit the generated medical image data to the PACS. The PACS is a system configured to manage various types of medical image data. The PACS includes a PACS server 40.

For example, the PACS server 40 is configured to receive the patient information transmitted thereto from the HIS server 10 and to manage the received patient information. Further, for example, the PACS server 40 is configured to receive the medical image data transmitted thereto from the medical image diagnosis apparatus 30 and to manage the received medical image data so as to be kept in correspondence with the patient information. Further, the PACS server 40 is configured to transmit medical image data corresponding to an obtainment request from the medical report generating device 100, to the medical report generating device 100. In this situation, the medical image data saved in the PACS server 40 has appended thereto the additional information such as a patient ID, an examination ID, an apparatus ID, an image series ID, and the like. For this reason, the medical report generating device 100 is able to obtain necessary patient information from the PACS server 40, by conducting a search while using a patient ID, or the like. Further, the medical report generating device 100 is able to obtain necessary medical image data from the PACS server 40, by conducting a search while using a patient ID, an examination ID, an apparatus ID, an image series ID, and/or the like.

Further, for example, the PACS server 40 is configured to receive the medical report and the examination execution information corresponding to the medical report transmitted thereto from the medical report generating device 100 and to manage the medical report and the examination execution information that were received, so as to be kept in correspondence with each other. The medical report is generated by the medical report generating device 100. As explained above, the medical report saved in the PACS server 40 is kept in correspondence with the examination execution information, while the examination execution information contains the examination ID, the patient ID, the examined site, the examination type, the date of the examination, and the like. Accordingly, the medical report generating device 100 is able to obtain necessary medical reports from the PACS server 40, by conducting a search while using a patient ID, an examination ID, and/or the like.

In the medical report generating device 100, a viewer (software) is installed, which is used for displaying, on a display thereof, an image (a medical image) based on medical image data or a medical report. In other words, the medical report generating device 100 is used as a viewer of the PACS (hereinafter, “image display viewer”).

For example, the medical report generating device 100 is provided for a radiology department and is operated by an image interpreting doctor. The image interpreting doctor performs image interpretation by using medical images displayed by the medical report generating device 100 or the like.

For example, when the examination type is a CT examination, while the examined site is the lungs, the image interpreting doctor measures the size of a tumor by using a medical image displayed by the medical report generating device 100. More specifically, by using the medical image, the image interpreting doctor measures the length of a line segment (e.g., the major axis) of the tumor being a primary lesion, as a measurement value. After that, the image interpreting doctor generates a medical report in which observations and the like including the measurement value (the value of the major axis of the tumor being the primary lesion) are written.

As another example, when the examination type is a CT examination, while the examined site is the liver, the image interpreting doctor measures the level of darkness of an image of the liver by using a medical image displayed by the medical report generating device 100. For example, by using the medical image, the image interpreting doctor measures a CT value of the image of the liver as a measurement value, to use the CT value for assessment of a fatty liver. After that, the image interpreting doctor generates a medical report in which observations and the like including the measurement value (the CT value of the image of the liver) are written.

In this situation, examples of the various types of servers illustrated in FIG. 1 include a clinical application server 50, a medical dictionary server 60, and a specimen examination server 70. The observations written in the medical report may include other parameters in addition to the measurement value (e.g., the value of the major axis of the tumor being the primary lesion).

For example, the clinical application server 50 has installed therein a clinical application (a computer program). The medical report generating device 100 is capable of reading the clinical application. The clinical application performs calculations on the basis of measurement values and obtains results of the calculations as clinical analysis results. For example, by using the clinical application read by the medical report generating device 100, the image interpreting doctor calculates the volume of the tumor on the basis of the measurement value (the value of the major axis of the tumor being the primary lesion) and obtains the calculated volume of the tumor as a clinical analysis result. After that, the image interpreting doctor generates a medical report in which observations and the like including the measurement value (the value of the major axis of the tumor being the primary lesion) and the clinical analysis result (the volume of the tumor) are written.

For example, as a medical dictionary, the medical dictionary server 60 has stored therein information keeping each of different sites with measurement values and categories. The medical report generating device 100 is capable of reading the medical dictionary. Examples of the categories include stages of cancer. For example, by referring to the medical dictionary read by the medical report generating device 100, the image interpreting doctor determines a stage of the cancer corresponding to the measurement value (the value of the major axis of the tumor being the primary lesion). After that, the image interpreting doctor generates a medical report in which observations and the like including the measurement value (the value of the major axis of the tumor being the primary lesion) and the stage of the cancer are written.

For example, the specimen examination server 70 has stored therein, as specimen examinations, vital data including the weight, a blood pressure value, a pulse rate, and the body temperature of each patient, as well as information about a result of a blood test, and the like, so as to be kept in correspondence with a patient ID, an examination ID and the like. Accordingly, the medical report generating device 100 is able to obtain necessary specimen examinations from the specimen examination server 70, by conducting a search while using a patient ID, an examination ID, and/or the like. For example, by using the medical report generating device 100, the image interpreting doctor obtains a specimen examination (e.g., a body temperature value) corresponding to a patient ID, an examination ID, and/or the like. After that, the image interpreting doctor generates a medical report in which observations and the like including the measurement value (the value of the major axis of the tumor being the primary lesion) and the specimen examination (the body temperature value) are written.

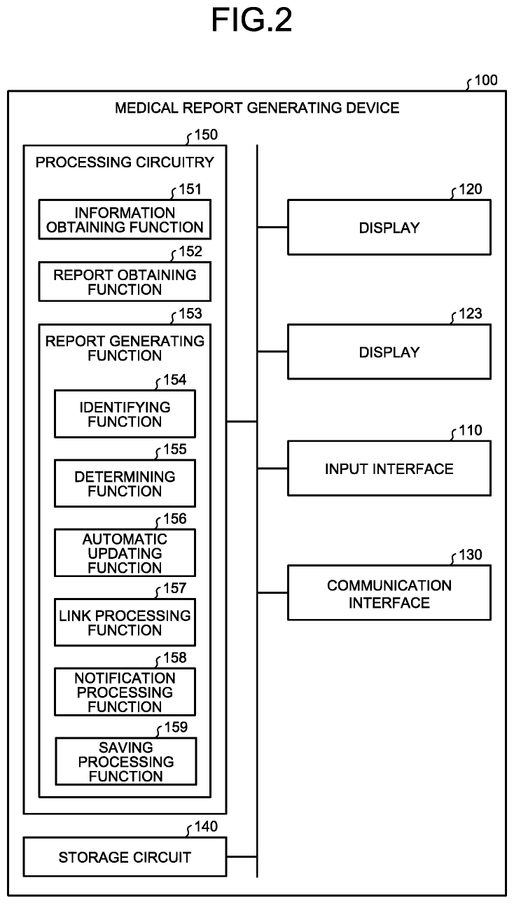

Next, details of the medical report generating device 100 according to the present embodiment will be explained. FIG. 2 is a diagram illustrating an exemplary configuration of the medical report generating device 100 according to the present embodiment. As illustrated in FIG. 2, the medical report generating device 100 includes an input interface 110, displays 120 and 123, a communication interface 130, a storage circuit 140, and processing circuitry 150.

The input interface 110 includes a pointing device such as a mouse, a keyboard, and the like and is configured to receive inputs of various types of operations performed on the medical report generating device 100 from an operator and to transfer instructions and setting information received from the operator to the processing circuitry 150. For example, the input interface 110 receives a medical image obtainment request, an image processing condition, and the like, from the operator.

The displays 120 and 123 are monitors referenced by the operator and are configured, under control of the processing circuitry 150, to display image data for the operator and to display a Graphical User Interface (GUI) used for receiving various types of instructions and various types of settings from the operator via the input interface 110. The communication interface 130 may be a Network Interface Card (NIC) or the like and is configured to communicate with other apparatuses.

As explained above, the image interpreting doctor performs the image interpretation by using the medical image data obtained during an examination, and the like and generates the medical report. For example, when having performed an examination on a patient at this time to observe progress, the image interpreting doctor generates a medical report (a current medical report). For example, the display 123 serves as a display unit configured to display the current medical report. For example, the display 120 serves as a display unit configured to display a list of medical reports (past medical reports) generated when examinations were performed on the patient in the past and to display a past medical report selected from the list.

The storage circuit 140 is a storage device that may be, for example, a semiconductor memory element such as a Random Access Memory (RAM), a Flash Memory, or the like, or a hard disk, an optical disk, or the like.

The processing circuitry 150 is configured to control constituent elements of the medical report generating device 100. For example, as illustrated in FIG. 2, the processing circuitry 150 executes an information obtaining function 151, a report obtaining function 152, and a report generating function 153. In this situation, for example, the processing functions executed by the constituent elements of the processing circuitry 150, namely, the information obtaining function 151, the report obtaining function 152, and the report generating function 153, are recorded in the storage circuit 140 in the form of computer-executable programs. The processing circuitry 150 is a processor configured to realize the functions corresponding to the programs by reading and executing the programs from the storage circuit 140. In other words, the processing circuitry 150 that has read the programs have the functions illustrated within the processing circuitry 150 in FIG. 2. In this situation, the information obtaining function 151 and the report obtaining function 152 are examples of an obtaining unit. The report generating function 153 is an example of a generating unit.

Further, the report generating function 153 includes an identifying function 154, a determining function 155, an automatic updating function 156, a link processing function 157, a notification processing function 158, and a saving processing function 159. The identifying function 154 is an example of an identifying unit. The determining function 155 is an example of a determining unit. The automatic updating function 156 is an example of an automatic updating unit. The link processing function 157 is an example of a link processing unit. The notification processing function 158 is an example of a notification processing unit. The saving processing function 159 is an example of a saving processing unit.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The processors realize the functions by reading and executing the programs saved in a storage circuit. In this situation, instead of saving the programs in the storage circuit, it is also acceptable to directly incorporate the programs in the circuits of the processors. In that situation, the processors realize the functions by reading and executing the programs incorporated in the circuits thereof. Further, the processors in the present embodiment do not each necessarily have to be structured as a single circuit. It is also acceptable to structure one processor by combining together a plurality of independent circuits so as to realize the functions thereof.

An overall configuration of the medical report generating system including the medical report generating device 100 according to the present embodiment has thus been explained. The medical report generating device 100 according to the present embodiment structured as described above is configured to perform processes described below, for the purpose of lessening the burden related to the generation of the medical reports. At first, in the medical report generating device 100 according to the present embodiment, the processing circuitry 150 obtains a medical report generated in the past. Further, on the basis of the past medical report, the processing circuitry 150 generates a template for a current medical report and causes a display unit (e.g., the display 123) to display update candidate items that need to be updated in the current medical report in a recognizable manner, together with the template. In this manner, because the medical report generating device 100 according to the present embodiment is configured to cause the display unit to display, in the recognizable manner, the update candidate items that need to be updated in the current medical report, the image interpreting doctor does not have to determine for himself/herself the update candidate items in the current medical report. It is therefore possible to lessen the burden that might be imposed on the image interpreting doctor when he/she had to determine the update candidate items for himself/herself. In other words, the medical report generating device 100 according to the present embodiment is able to make the updating work efficient. Further, the medical report generating device 100 according to the present embodiment is able to prevent the image interpreting doctor from overlooking the update candidate items while generating the current medical report.

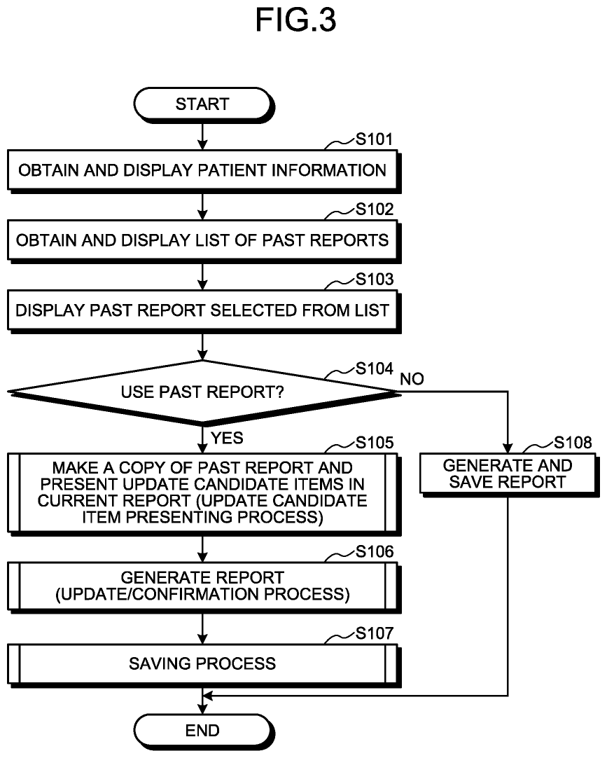

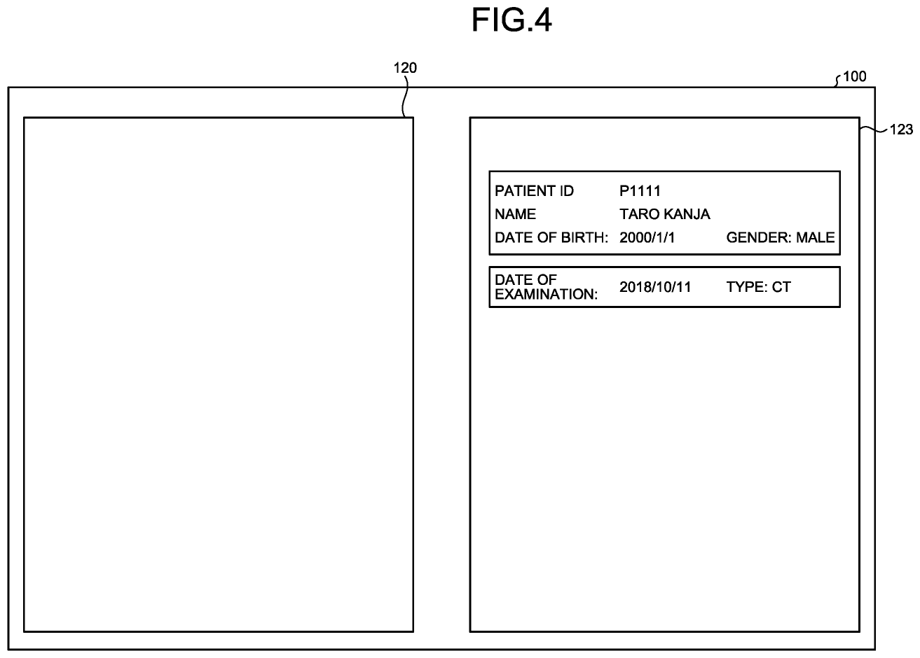

FIG. 3 is a flowchart illustrating a procedure in processes performed by the medical report generating device 100 according to the present embodiment. FIGS. 4 to 7 are drawings for explaining processes performed by the medical report generating device 100 according to the present embodiment.

Step S101 in FIG. 3 is a step executed as a result of the processing circuitry 150 invoking a program corresponding to the information obtaining function 151 from the storage circuit 140.

At step S101, the information obtaining function 151 obtains patient information (the patient ID, the patient's name (the family name and the given name), the age (the date of birth), the gender, the height, the weight, the blood type, etc.) from the PACS server 40, by conducting a search while using the patient ID contained in the most recent examination execution information (the examination ID, the patient ID, the examined site, the examination type, and the date of the examination, etc.). Further, as illustrated in FIG. 4, from among the obtained patient information, the information obtaining function 151 causes the display 123 to display, for example, the patient ID “P1111”, the name “Taro Kanja”, the date of birth “Jan. 1, 2000”, and the gender “male”. Further, as illustrated in FIG. 4, from among the most recent examination execution information, the information obtaining function 151 causes the display 123 to display, for example, the date of the examination “Oct. 11, 2018” and the examination type “CT examination”.

Steps S102 and S103 in FIG. 3 are steps executed as a result of the processing circuitry 150 invoking a program corresponding to the report obtaining function 152 from the storage circuit 140.

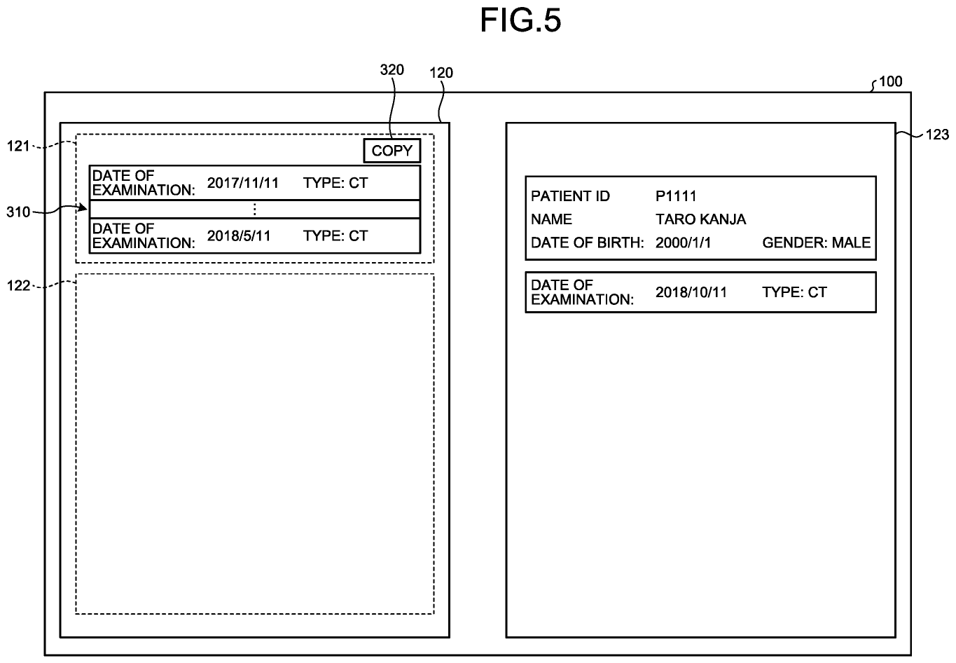

At step S102, the report obtaining function 152 obtains a plurality of pieces of examination execution information respectively corresponding to a plurality of past medical reports, from the PACS server 40, by conducting a search while using the patient ID contained in the most recent examination execution information. Further, as illustrated in FIG. 5, the report obtaining function 152 causes the display 120 to display, in a display region 121, the obtained plurality of pieces of examination execution information as a list 310 of the plurality of past medical reports. For example, as the list 310, the report obtaining function 152 causes the display 120 to display, in the display region 121, the date of the examination and the examination type of each of the plurality of pieces of examination execution information. Further, as illustrated in FIG. 5, the report obtaining function 152 causes the display 120 to display, in the display region 121, a copy button 320 used for making a copy of one of the past medical reports selected from the list 310.

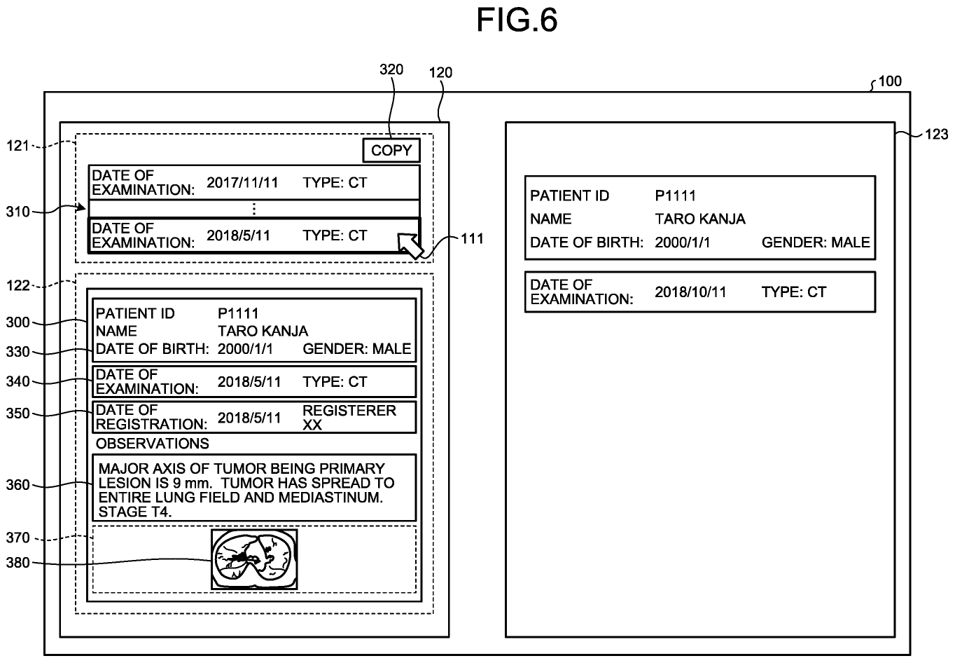

At step S103, the report obtaining function 152 causes the display 120 to display, in a display region 122, the past medical report selected from the list 310. For example, as illustrated in FIG. 6, the image interpreting doctor operates the input interface 110 (FIG. 2) to perform a click operation while placing a cursor 111 on one of the plurality of pieces of examination execution information indicated in the list 310 displayed in the display region 121 of the display 120. As a result, the one piece of examination execution information is selected. For example, the selected piece of examination execution information represents an examination performed on the patient immediately before the examination at this time. In the display region 121 of the display 120, the date of the examination “May 11, 2018” and the examination type “CT examination” are displayed. Further, as illustrated in FIG. 6, the report obtaining function 152 obtains a past medical report 300 kept in correspondence with the selected piece of examination execution information from the PACS server 40 and further causes the display 120 to display, in the display region 122, the obtained past medical report 300.

As illustrated in FIG. 6, the past medical report 300 contains a patient information display section 330, an examination execution information display section 340, and a registered information display section 350. For example, from among the patient information, the patient information display section 330 displays the patient ID “P1111”, the name “Taro Kanja”, the date of birth “Jan. 1, 2000”, and the gender “male”. For example, from among the selected piece of examination execution information, the examination execution information display section 340 displays the date of the examination “May 11, 2018” and the examination type “CT examination”. The registered information display section 350 displays a registration date “May 11, 2018” on which the past medical report 300 was saved (registered) into the PACS server 40 and the name “XX” of the image interpreting doctor (a registerer) who generated the past medical report 300.

Further, as illustrated in FIG. 6, the past medical report 300 further contains an observation display section 360. For example, the observation display section 360 displays, with text data, observations “MAJOR AXIS OF TUMOR BEING PRIMARY LESION IS 9 mm. TUMOR HAS SPREAD TO ENTIRE LUNG FIELD AND MEDIASTINUM. STAGE T4.” that were made by the image interpreting doctor while interpreting medical images and the like.

Further, as illustrated in FIG. 6, the past medical report 300 further contains a multimedia data display section 370. For example, as multimedia data, the multimedia data display section 370 displays a key image 380 generated during a past examination performed on the patient. The key image 380 may be a tomographic image indicating a lesion site in an easy-to-see manner in a certain slice position or may be a tomographic image indicating a site to which particular attention should be paid in a certain slice position. The key image 380 is represented by medical image data obtained from the PACS server 40 by the medical report generating device 100. The medical image data selected as the key image 380 is converted into a general-purpose image format such as a Joint Photographic Experts Group (JPEG) format or a bitmap format and is displayed while being pasted on the past medical report 300.

Although the multimedia data display section 370 displays the single key image 380, possible embodiments are not limited to this example. For instance, the multimedia data display section 370 may display a plurality of key images taken in mutually-different slice positions.

Further, the multimedia data display section 370 displays the key image 380 as the multimedia data; however, possible embodiments are not limited to this example. For instance, the multimedia data display section 370 may display, besides the key image 380, a key moving image, a Portable Document Format (PDF) file in which a medical article is written, multimedia data such as a moving image file or an audio file, and/or the like. To any of these pieces of multimedia data, link information used for invoking the piece of multimedia data from the PACS server 40 may be appended. For example, when a click operation is performed on the link information, a corresponding piece of multimedia data is invoked from the PACS server 40, so as to be displayed by an image display viewer, a moving image viewer, a PDF application, or the like.

Steps S104 through S108 in FIG. 3 are steps executed as a result of the processing circuitry 150 invoking a program corresponding to the report generating function 153 from the storage circuit 140.

At step S104, the report generating function 153 monitors whether or not the image interpreting doctor is to use a copy of the past medical report 300 when generating the current medical report. More specifically, the report generating function 153 monitors whether or not the copy button 320 displayed in the display region 121 of the display 120 is operated while the past medical report 300 is displayed in the display region 122 of the display 120.

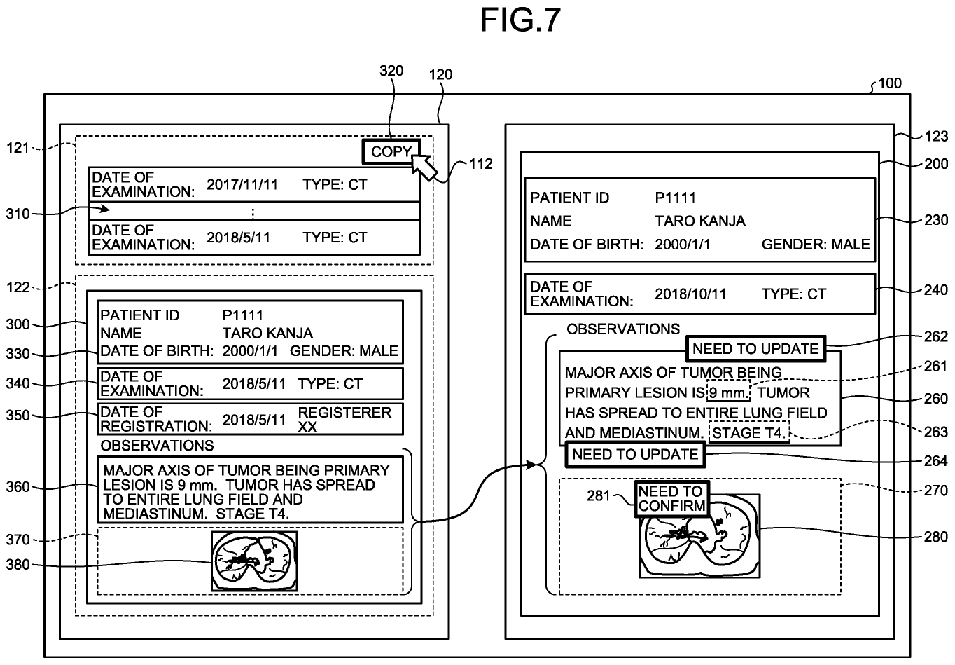

For example, as illustrated in FIG. 7, when the image interpreting doctor is to use a copy of the past medical report 300 when generating the current medical report (step S104: Yes), the image interpreting doctor operates the input interface 110 (FIG. 2) and performs a click operation while placing a cursor 112 on the copy button 320 displayed in the display region 121 of the display 120. In this situation, at step S105 (an update candidate item presenting process), the report generating function 153 generates a template for a current medical report 200 by making a copy of the past medical report 300 displayed in the display region 122 of the display 120 and further causes the display 123 to display the update candidate items that need to be updated in the current medical report 200 in a recognizable manner, together with the template for the current medical report 200. In this situation, the template for the current medical report 200 is the medical report 200 before being saved and is an example of the first medical report.

As illustrated in FIG. 7, the template for the current medical report 200 contains a patient information display section 230 and an examination execution information display section 240. The patient information display section 230 displays the patient information obtained at step S101. For example, from among the patient information obtained at step S101, the patient information display section 230 displays the patient ID “P1111”, the name “Taro Kanja”, the date of birth “Jan. 1, 2000”, and the gender “male”. The examination execution information display section 240 displays the most recent examination execution information. For example, from among the most recent examination execution information, the examination execution information display section 240 displays the date of the examination “Oct. 11, 2018” and the examination type “CT examination”.

Also, as illustrated in FIG. 7, the template for the current medical report 200 further contains an observation display section 260. The observation display section 260 displays data obtained by copying the observations “MAJOR AXIS OF TUMOR BEING PRIMARY LESION IS 9 mm. TUMOR HAS SPREAD TO ENTIRE LUNG FIELD AND MEDIASTINUM. STAGE T4.”, which is the text data displayed in the observation display section 360 of the past medical report 300 and subsequently making the update candidate items in the observations (the text data) recognizable.

More specifically, the character string “9 mm” in the observations displayed in the observation display section 260 is an update candidate item that needs to be updated in the current medical report 200. To the character string “9 mm”, link information 261 making the update candidate item recognizable is appended. The link information 261 is link information used for linking to the PACS server 40. Further, displayed in the vicinity of the character string “9 mm” in the observation display section 260 is a message 262 “NEED TO UPDATE” making the update candidate item recognizable. The message 262 “NEED TO UPDATE” is a message prompting the image interpreting doctor to update the character string “9 mm”. Details of this process will be explained later.

Further, the character string “STAGE T4” in the observations displayed in the observation display section 260 is another update candidate item that needs to be updated in the current medical report 200. To the character string “STAGE T4”, link information 263 making the update candidate item recognizable is appended. The link information 263 is link information for linking to the medical dictionary server 60. Further, displayed in the vicinity of the character string “STAGE T4” in the observation display section 260 is a message 264 “NEED TO UPDATE” making the update candidate item recognizable. The message 264 “NEED TO UPDATE” is a message for prompting the image interpreting doctor to update the character string “STAGE T4”. Details of this process will be explained later.

Also, as illustrated in FIG. 7, the template for the current medical report 200 further contains a multimedia data display section 270. In the multimedia data display section 270, the key image 380 displayed in the multimedia data display section 370 of the past medical report 300 is copied, and subsequently, a key image 280 generated during the examination performed on the patient at this time is displayed in place of the key image 380, as a result of an automatic update (explained later). Further, displayed either on the inside or in the vicinity of the key image 280 in the multimedia data display section 270 is a message 281 “NEED TO CONFIRM” making the update candidate item recognizable. The message 281 “NEED TO CONFIRM” is a message for prompting the image interpreting doctor to confirm the key image 280. Details of this process will be explained later.

In the present example, at steps S102 through S105, when the image interpreting doctor operates the copy button 320, the report generating function 153 makes the copy of the past medical report 300 displayed in the display region 122 of the display 120; however, possible embodiments are not limited to this example. For instance, at step S105, the report generating function 153 may automatically make a copy of the past medical report 300 (which hereinafter will be referred to as an “automatic copy process”), by conducting a search while using the examination ID, the patient ID, the examined site, the examination type, the date of the examination, and the like contained in the most recent examination execution information. When the automatic copy process is performed, processes at steps S102 through S104 will not be performed.

For example, the report generating function 153 is able to obtain the past medical report 300 from the PACS server 40, by conducting the search while using the examination ID, the patient ID, the examined site, the examination type, the date of the examination, and the like contained in the most recent examination execution information. More specifically, as explained above, the past medical reports are saved in the PACS server 40 together with the examination execution information corresponding to the past medical reports. Accordingly, by conducting the search while using the examination ID, the patient ID, the examined site, the examination type, the date of the examination, and the like contained in the most recent examination execution information, the report generating function 153 is able to obtain, from the PACS server 40, such a medical report (the past medical report 300) among the past medical reports saved in the PACS server 40 that corresponds to the examination ID, the patient ID, the examined site, and the examination type contained in the most recent examination execution information and that also was generated at the time of an examination performed immediately before the date of the examination contained in the most recent examination execution information. Further, the report obtaining function 152 makes a copy of the obtained past medical report 300.

In this situation, the image interpreting doctor may also select whether or not the automatic copy process is to be executed. For example, in the display region 121 of the display 120, a check box used for cancelling the execution of the automatic copy process is displayed. When the image interpreting doctor operates the input interface 110 to check in the check box, the report generating function 153 deactivates the execution of the automatic copy process.

Subsequently, at step S106 (an update/confirmation process), the report generating function 153 generates the current medical report 200 by updating only the update candidate items according to operations performed by the image interpreting doctor. Details of this process will be explained later.

Further, at step S107 (a saving process), when the image interpreting doctor issues a save instruction by using the input interface 110, the report generating function 153 converts the data format of the observations displayed in the observation display section 260 into text data (i.e., removes the link information) and generates the current medical report. The generated current medical report 200 is newly provided with a registered information display section containing a registration date “Oct. 11, 2018” on which the current medical report 200 is saved (registered) into the PACS server 40 and the name “XX” of the image interpreting doctor (the registerer) who generated the current medical report 200. Further, the report generating function 153 saves (registers) the generated current medical report 200 into the PACS server 40. More specifically, the report generating function 153 transmits the generated current medical report 200 and the examination execution information corresponding to the current medical report 200 to the PACS server 40. In this situation, the PACS server 40 receives the current medical report 200 and the examination execution information corresponding to the current medical report 200 transmitted thereto from the medical report generating device 100 and further manages the current medical report 200 and the examination execution information that were received, so as to be kept in correspondence with each other. Details of this process will be explained later.

In contrast, when the image interpreting doctor is not to use a copy of the past medical report 300 when generating the current medical report (step S104: No), the image interpreting doctor operates the input interface 110 (FIG. 2) and performs a click operation while placing a cursor on a “CREATE NEW” button (not illustrated) displayed in the display region 121 of the display 120. In that situation, at step S108, the report generating function 153 generates a template for the current medical report in which the observation display section 260 and the multimedia data display section 270 are blank and further causes the display 123 to display the generated template. The image interpreting doctor generates a current medical report by writing observations in the observation display section 260, pasting a key image in the multimedia data display section 270, and the like. The generated current medical report 200 is newly provided with a registered information display section containing a registration date “Oct. 11, 2018” on which the current medical report 200 is saved (registered) into the PACS server 40 and the name “XX” of the image interpreting doctor (the registerer) who generated the current medical report 200. Further, when the image interpreting doctor issues a save instruction by using the input interface 110, the report generating function 153 saves the generated current medical report. More specifically, the report generating function 153 transmits the generated current medical report and the examination execution information corresponding to the current medical report, to the PACS server 40. In that situation, the PACS server 40 receives the current medical report and the examination execution information corresponding to the current medical report transmitted thereto from the medical report generating device 100 and further manages the current medical report and the examination execution information that were received, so as to be kept in correspondence with each other.

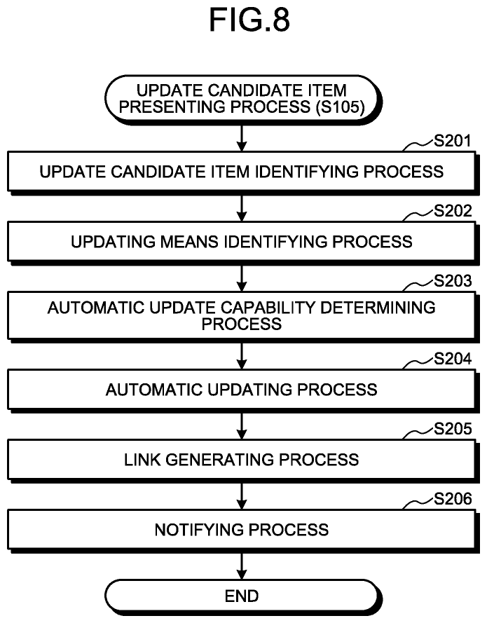

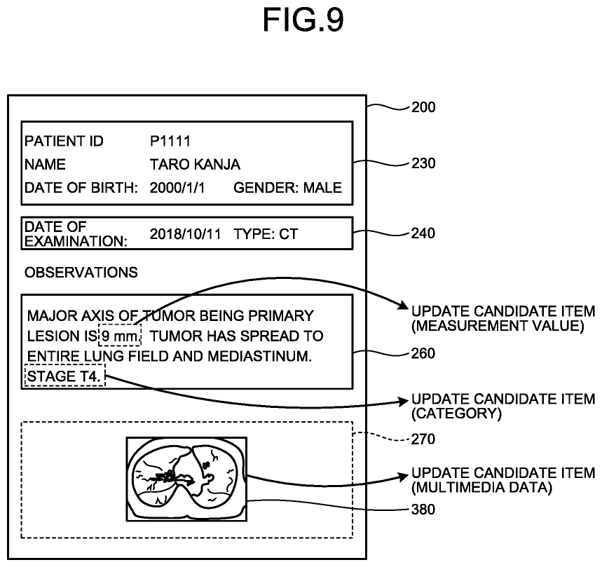

FIG. 8 is a flowchart illustrating a procedure in the update candidate item presenting process (step S105) according to the present embodiment. FIGS. 9 to 13 are drawings for explaining the update candidate item presenting process (step S105) according to the present embodiment.

Step S201 in FIG. 8 is a step executed as a result of the processing circuitry 150 invoking a program corresponding to the identifying function 154 of the report generating function 153 from the storage circuit 140. At step S201, the identifying function 154 identifies the update candidate items that need to be updated in the current medical report 200, from within the data (the text data and the multimedia data) contained in the template for the current medical report 200. In other words, the identifying function 154 identifies whether or not the template for the current medical report 200 contains one or more of measurement values, clinical analysis results, categories, and multimedia data.

The identifying function 154 at first identifies whether or not the template for the current medical report 200 contains one or more of measurement values, clinical analysis results, and categories. For example, the identifying function 154 identifies whether or not the observations “MAJOR AXIS OF TUMOR BEING PRIMARY LESION IS 9 mm. TUMOR HAS SPREAD TO ENTIRE LUNG FIELD AND MEDIASTINUM. STAGE T4.” represented by the text data in the observation display section 260 in the template for the current medical report 200 contain one or more of measurement values, clinical analysis results, and categories. In this situation, examples of techniques that can be used for detecting the presence/absence of writing of one or more of measurement values, clinical analysis results, and categories include an analysis using natural language processing. By performing the natural language processing, the identifying function 154 is able to identify information indicating, for example, whether a certain character string represents writing of a measurement value of a lesion site or represents writing of a category.

For example, as illustrated in FIG. 9, from the character string “MAJOR AXIS OF TUMOR . . . IS 9 mm” in the observations in the observation display section 260, the identifying function 154 identifies that “9 mm” is writing about a measurement value of the tumor. In other words, the identifying function 154 identifies the measurement value “9 mm” as an update candidate item that needs to be updated in the current medical report 200.

For example, as illustrated in FIG. 9, from the character string “STAGE T4.” in the observations in the observation display section 260, the identifying function 154 identifies “STAGE T4” as writing about a category (the stage of the cancer). In other words, the identifying function 154 identifies the category “STAGE T4” as an update candidate item that needs to be updated in the current medical report 200.

Subsequently, the identifying function 154 identifies whether or not the template for the current medical report 200 contains multimedia data. In this situation, the identifying function 154 checks to see whether multimedia data is present or absent in the multimedia data display section 270 in the template for the current medical report 200.

For example, as illustrated in FIG. 9, the key image 380 is pasted as multimedia data in the multimedia data display section 270. Accordingly, the identifying function 154 identifies the key image 380 as an update candidate item that needs to be updated in the current medical report 200.

In the text data, the identifying function 154 is configured to determine measurement values, clinical analysis results, categories, and the like as update candidate items; however, possible embodiments are not limited to this example. For instance, in the text data, the identifying function 154 may determine writing about a transition such as “enlarging” or “reducing in size” as an update candidate item.

Step S202 in FIG. 8 is a step executed as a result of the processing circuitry 150 invoking the program corresponding to the identifying function 154 of the report generating function 153 from the storage circuit 140. At step S202, the identifying function 154 refers to a table and identifies an updating means for implementing an updating method to update the identified update candidate items (the measurement value “9 mm”, the category “STAGE T4”, and the key image 380).

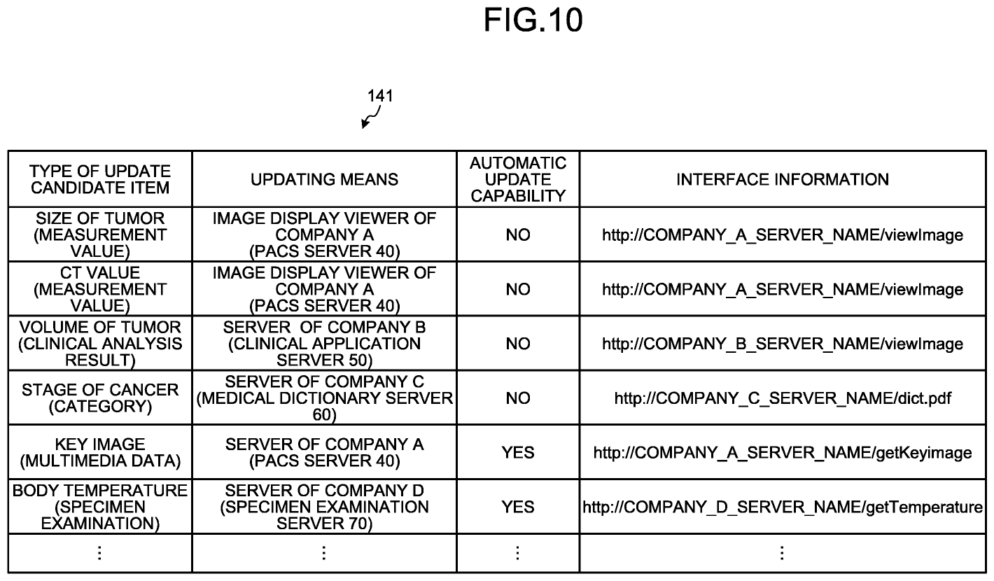

More specifically, the storage circuit 140 includes a table 141 illustrated in FIG. 10. As illustrated in FIG. 10, the table 141 stores therein information keeping, in correspondence with one another, types of update candidate items, updating means for updating the update candidate items, automatic update capability information indicating whether or not each updating means is automatically updatable, and link destinations (interface information) to the updating means.

For example, the table 141 stores therein information keeping, in correspondence with one another, the type “the size of the tumor (a measurement value)” of an update candidate item, an updating means “the image display viewer of Company A (the PACS server 40)” for updating the update candidate item, automatic update capability information “NO (not updatable)” of the updating means, and a link destination (the interface information) to the updating means. As another example, the table 141 stores therein information keeping, in correspondence with one another, the type “a CT value (a measurement value)” of an update candidate item, an updating means “the image display viewer of Company A (the PACS server 40)” for updating the update candidate item, automatic update capability information “NO (not updatable)” of the updating means, and a link destination (the interface information) to the updating means. For example, the identifying function 154 refers to the table 141 and identifies the updating means “the image display viewer of Company A (the PACS server 40)” corresponding to the type “the size of the tumor (a measurement value)” of the update candidate item, as the updating means for updating the update candidate item (the measurement value “9 mm”) identified from the character string “MAJOR AXIS OF TUMOR IS 9 mm.” in the observations in the observation display section 260.

As yet another example, the table 141 stores therein information keeping, in correspondence with one another, the type “the volume of the tumor (a clinical analysis result)” of an update candidate item, an updating means “the server of Company B (the clinical application server 50)” for updating the update candidate item, automatic update capability information “NO (not updatable)” of the updating means, and a link destination (the interface information) to the updating means. As yet another example, the table 141 stores therein information keeping, in correspondence with one another, the type “the stage of the cancer (a category)” of an update candidate item, an updating means “the server of Company C (the medical dictionary server 60)” for updating the update candidate item, automatic update capability information “NO (not updatable)” of the updating means, and a link destination (the interface information) to the updating means. For example, by referring to the table 141, the identifying function 154 identifies the updating means “the server of Company C (the medical dictionary server 60)” corresponding to the type “the stage of the cancer (a category)” of the update candidate item, as an updating means for updating the update candidate item (the category “STAGE T4”) identified from the character string “STAGE T4” in the observations in the observation display section 260.

As yet another example, the table 141 stores therein information keeping, in correspondence with one another, the type “a key image (multimedia data)” of an update candidate item, an updating means “the server of Company A (the PACS server 40)” for updating the update candidate item, automatic update capability information “YES (updatable)” of the updating means, and a link destination (the interface information) to the updating means. As yet another example, the table 141 stores therein information keeping, in correspondence with one another, the type “the body temperature (a specimen examination)” of an update candidate item, an updating means “the server of Company D (the specimen examination server 70)” for updating the update candidate item, automatic update capability information “YES (updatable)” of the updating means, and a link destination (the interface information) to the updating means. For example, by referring to the table 141, the identifying function 154 identifies the updating means “the server of Company A (the PACS server 40)” corresponding to the type “a key image (multimedia data)” of the update candidate item, as the updating means for updating the update candidate item (the key image 380) identified from the multimedia data in the multimedia data display section 270.

Step S203 in FIG. 8 is a step executed as a result of the processing circuitry 150 invoking a program corresponding to the determining function 155 of the report generating function 153 from the storage circuit 140. At step S203, the determining function 155 refers to the table 141 and determines whether or not the update candidate items (the measurement value “9 mm”, the category “STAGE T4”, and the key image 380) identified in the current medical report 200 are automatically updatable.

For example, in the table 141, the automatic update capability information corresponding to the type “the size of the tumor (a measurement value)” of the update candidate item and to the updating means “the image display viewer of Company A (the PACS server 40)” indicates “NO (not updatable)”. In this situation, the determining function 155 refers to the table 141 and determines that the update candidate item (the measurement value “9 mm”) identified by the identifying function 154 is not automatically updatable.

As another example, in the table 141, the automatic update capability information corresponding to the type “the stage of the cancer (a category)” of the update candidate item and to the updating means “the server of Company C (the medical dictionary server 60)” indicates “NO (not updatable)”. In that situation, the determining function 155 refers to the table 141 and determines that the update candidate item (the category “STAGE T4”) identified by the identifying function 154 is not automatically updatable.

As yet another example, in the table 141, the automatic update capability information corresponding to the type “a key image (multimedia data)” of the update candidate item and to the updating means “the server of Company A (the PACS server 40)” indicates “YES (updatable)”. In that situation, the determining function 155 refers to the table 141 and determines that the update candidate item (the key image 380) identified by the identifying function 154 is automatically updatable.

Step S204 in FIG. 8 is a step executed as a result of the processing circuitry 150 invoking a program corresponding to the automatic updating function 156 of the report generating function 153 from the storage circuit 140. At step S204, when the determining function 155 determines that the update candidate item (the key image 380) identified by the identifying function 154 is automatically updatable, the automatic updating function 156 automatically updates the update candidate item (the key image 380) by using the updating means “the server of Company A (the PACS server 40)” corresponding to the update candidate item (the key image 380).

For example, by conducting a search while using the patient ID, the examination ID, an image ID, and/or the like, the automatic updating function 156 is able to obtain, from the PACS server 40, a key image (medical image data) that was generated during the examination performed at this time and that corresponds to the update candidate item (the key image 380) identified by the identifying function 154. More specifically, as explained above, the medical image data saved in the PACS server 40 has additional information appended thereto such as a patient ID, an examination ID, an image ID, and the like. Further, as explained above, the image ID contains, for example, the site of the examined subject (the patient) that was imaged, the image generation time, the slick thickness, the slice position, and the like. Accordingly, by conducting a search while using the patient ID of the patient, the examination ID of the examination performed on the patient at this time, the image ID of the key image 380 generated during the examination performed on the patient in the past, and the like, the automatic updating function 156 is able to obtain, from the PACS server 40, a key image (medical image data) in a slice position corresponding to the slice position of the key image 380 generated during the past examination, from within the medical image data generated during the examination at this time.

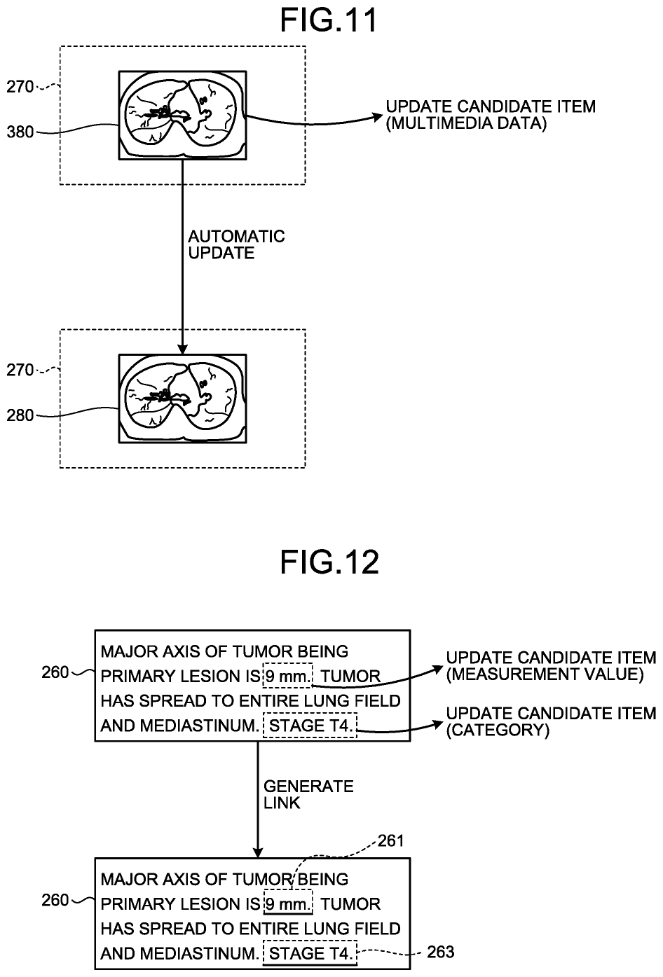

Further, as illustrated in FIG. 11, the automatic updating function 156 updates the update candidate item (the key image 380) identified by the identifying function 154 with the key image 280 obtained from the PACS server 40. In other words, as a result of the automatic update, the multimedia data display section 270 displays the key image 280 generated during the examination performed on the patient at this time, in place of the update candidate item (the key image 380) identified by the identifying function 154.

The automatic updating function 156 is configured to obtain the key image generated during the examination performed at this time, on the basis of the key image 380 generated during the past examination according to the slice position; however, possible embodiments are not limited to this example. For instance, the automatic updating function 156 may obtain a key image generated during the examination performed at this time, on the basis of the key image 380 generated during the past examination according to the shape of the site rendered in the key image 380.

Step S205 in FIG. 8 is a step executed as a result of the processing circuitry 150 invoking a program corresponding to the link processing function 157 of the report generating function 153 from the storage circuit 140. At step S205, when the determining function 155 determines that the update candidate item identified by the identifying function 154 is not automatically updatable, the link processing function 157 appends, to the update candidate item, link information linking to an updating means corresponding to the update candidate item as a link destination.

For example, the determining function 155 determines that the update candidate item (the measurement value “9 mm”) identified by the identifying function 154 is not automatically updated. In that situation, as illustrated in FIG. 12, the link processing function 157 appends, to the update candidate item (the measurement value “9 mm”), link information 261 linking to the updating means “the image display viewer of Company A (the PACS server 40)” corresponding to the update candidate item (the measurement value “9 mm”) as a link destination. In other words, to the character string “9 mm” in the observations displayed in the observation display section 260, the link information 261 for linking to the PACS server 40 is appended. In this situation, the link processing function 157 displays the update candidate item (the measurement value “9 mm”) that needs to be updated in the current medical report 200, in a recognizable manner in the observation display section 260, by underlining the character string “9 mm” or changing the color of the letters in the character string “9 mm” from black to blue.

As another example, the determining function 155 determines that the update candidate item (the category “STAGE T4”) identified by the identifying function 154 is not automatically updatable. In that situation, as illustrated in FIG. 12, the link processing function 157 appends, to the update candidate item (the category “STAGE T4”), link information 263 linking to the updating means “the server of Company C (the medical dictionary server 60)” corresponding to the update candidate item (the category “STAGE T4”), as a link destination. In other words, to the character string “STAGE T4” in the observations displayed in the observation display section 260, the link information 263 for linking to the medical dictionary server 60 is appended. In this situation, the link processing function 157 displays the update candidate item (the category “STAGE T4”) that needs to be updated in the current medical report 200, in a recognizable manner in the observation display section 260, by underlining the character string “STAGE T4” or changing the color of the letters in the character string “STAGE T4” from black to blue.

Step S206 in FIG. 8 is a step executed as a result of the processing circuitry 150 invoking a program corresponding to the notification processing function 158 of the report generating function 153 from the storage circuit 140. At step S206, the notification processing function 158 displays a message indicating that an update is necessary with respect to the update candidate item to which the link information is appended.

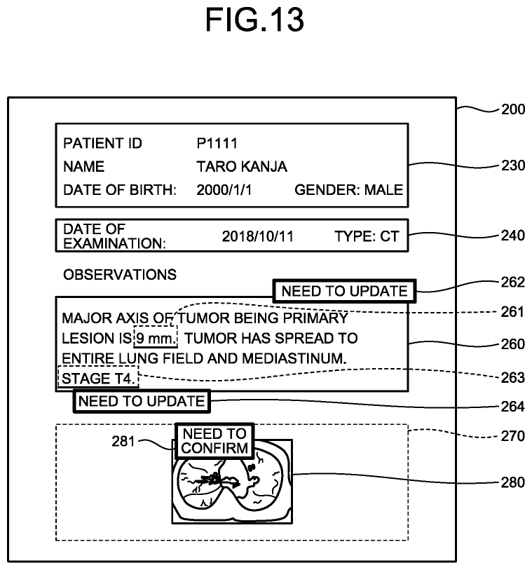

For example, as illustrated in FIG. 13, the notification processing function 158 displays a message 262 “NEED TO UPDATE” indicating that an update is necessary with respect to the update candidate item (the measurement value “9 mm”) to which the link information 261 is appended. In other words, in the vicinity of the character string “9 mm” in the observations in the observation display section 260, the message 262 “NEED TO UPDATE” is displayed to prompt the image interpreting doctor to update the character string “9 mm”. By displaying the message 262 “NEED TO UPDATE” in the vicinity of the character string “9 mm” in the observations in the observation display section 260, the notification processing function 158 displays the update candidate item (the measurement value “9 mm”) that needs to be updated in the current medical report 200, in a recognizable manner in the observation display section 260. That is to say, because the update candidate item (the measurement value “9 mm”) is an update candidate item that is not automatically updatable (an update candidate item to be manually updated) and requires an update (an update requiring item), the notification processing function 158 displays, in a recognizable manner in the observation display section 260, information indicating that the character string “9 mm” in the observation display section 260 needs to be updated by a manual update of the image interpreting doctor, as an update state of the update candidate item to be manually updated, by displaying the message 262 “NEED TO UPDATE”.

For example, as illustrated in FIG. 13, the notification processing function 158 displays a message 264 “NEED TO UPDATE” indicating that an update is necessary with respect to the update candidate item (the category “STAGE T4”) to which the link information 263 is appended. In other words, in the vicinity of the character string “STAGE T4” in the observations in the observation display section 260, the message 264 “NEED TO UPDATE” is displayed to prompt the image interpreting doctor to update the character string “STAGE T4”. By displaying the message 264 “NEED TO UPDATE” in the vicinity of the character string “STAGE T4” in the observations in the observation display section 260, the notification processing function 158 displays the update candidate item (the category “STAGE T4”) that needs to be updated in the current medical report 200, in a recognizable manner in the observation display section 260. That is to say, because the update candidate item (the category “STAGE T4”) is an update candidate item that is not automatically updatable (an update candidate item to be manually updated) and requires an update (an update requiring item), the notification processing function 158 displays, in a recognizable manner in the observation display section 260, information indicating that the character string “STAGE T4” in the observation display section 260 needs to be updated by a manual update of the image interpreting doctor, as an update state of the update candidate item to be manually updated, by displaying the message 264 “NEED TO UPDATE”.

For example, as illustrated in FIG. 13, with respect to the update candidate item (the key image 280) that was automatically updated, the notification processing function 158 displays the message 281 “NEED TO CONFIRM” indicating that a confirmation is necessary. In other words, displayed either on the inside or in the vicinity of the key image 280 in the multimedia data display section 270 is the message 281 “NEED TO CONFIRM” indicating that the key image 280 has been updated and prompting the image interpreting doctor to confirm the key image 280. By displaying the message 281 “NEED TO CONFIRM” either on the inside or in the vicinity of the key image 280 in the multimedia data display section 270, the notification processing function 158 displays, in a recognizable manner in the multimedia data display section 270, the update candidate item (the key image 280) that needs to be confirmed in the current medical report 200. That is to say, because the update candidate item (the key image 280) is an update candidate item that was automatically updated (an already-updated item), the notification processing function 158 displays, in a recognizable manner in the multimedia data display section 270, information indicating that the key image in the multimedia data display section 270 has automatically been updated and thus needs to be confirmed by the image interpreting doctor, as an update state of the update candidate item automatically updated, by displaying the message 281 “NEED TO CONFIRM”.

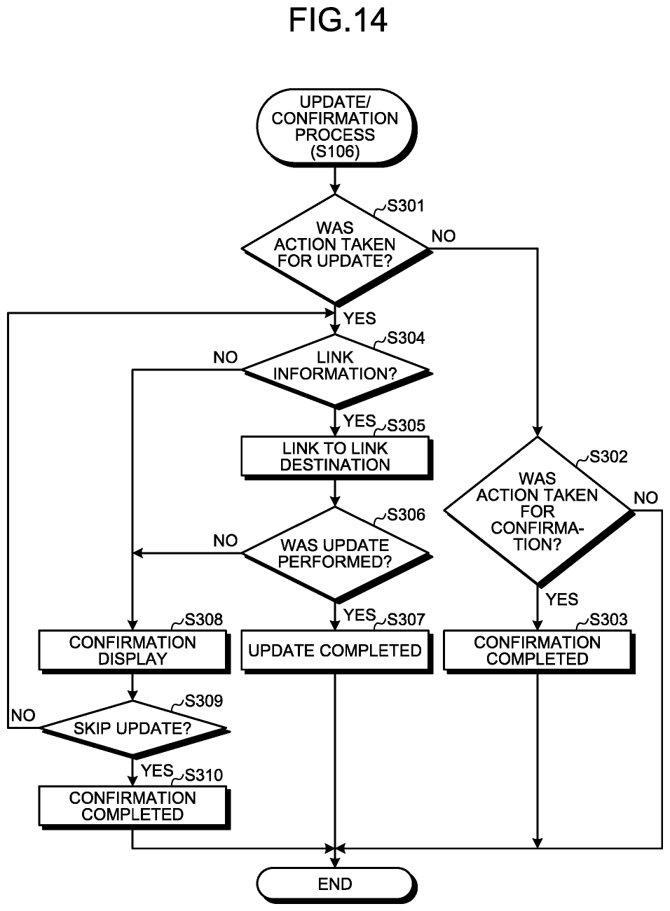

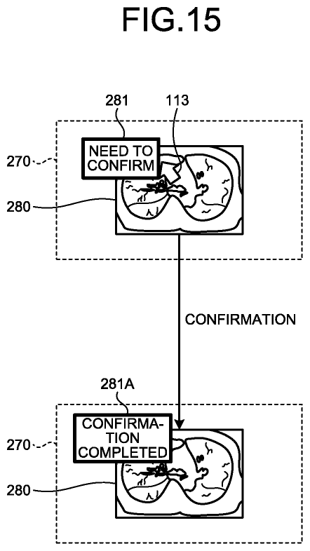

FIG. 14 is a flowchart illustrating a procedure in the update/confirmation process (step S106) according to the present embodiment. FIGS. 15 to 17 are drawings for explaining the update/confirmation process (step S106) according to the present embodiment.

Steps S301 through S304 and S306 through S310 in FIG. 14 are steps executed as a result of the processing circuitry 150 invoking a program corresponding to the notification processing function 158 of the report generating function 153 from the storage circuit 140. Step S305 in FIG. 14 is a step executed as a result of the processing circuitry 150 invoking a program corresponding to the link processing function 157 of the report generating function 153 from the storage circuit 140.

At step S301, the notification processing function 158 monitors whether or not an action for an update is taken. The action for an update may be, for example, the image interpreting doctor performing a click operation while placing a cursor on the link information 261 or 263 appended to the character string “9 mm” or “STAGE T4” in the observations in the observation display section 260, or on the message 262 or 264 “NEED TO UPDATE” displayed in the vicinity of the character string “9 mm” or “STAGE T4”, by using the input interface 110 (FIG. 2).

When no action for an update is taken (step S301: No), the notification processing function 158 monitors, at step S302, whether or not an action for a confirmation is taken. The action for a confirmation may be, for example, the image interpreting doctor performing a click operation while placing a cursor on the message 281 “NEED TO CONFIRM” displayed either on the inside or in the vicinity of the key image 280 in the multimedia data display section 270, by using the input interface 110.

When the action for a confirmation is taken (step S302: Yes), the notification processing function 158 displays, at step S303, a message indicating that the confirmation on the key image 280 by the image interpreting doctor is completed, in place of the message 281 “NEED TO CONFIRM”. For example, as illustrated in FIG. 15, the image interpreting doctor performs a click operation while placing a cursor 113 on the message 281 “NEED TO CONFIRM” by using the input interface 110. In that situation, in place of the message 281 “NEED TO CONFIRM”, the notification processing function 158 displays a message 281A “CONFIRMATION COMPLETED” indicating that the confirmation on the key image 280 by the image interpreting doctor is completed. In other words, in place of the message 281 “NEED TO CONFIRM”, the message 281A “CONFIRMATION COMPLETED” is displayed either on the inside or in the vicinity of the key image 280 in the multimedia data display section 270. That is to say, the notification processing function 158 displays, in a recognizable manner in the multimedia data display section 270, information indicating that the confirmation on the key image 280 in the multimedia data display section 270 is completed by the image interpreting doctor, as an update state of the update candidate item automatically updated, by displaying the message 281A “CONFIRMATION COMPLETED”.

When the action for a confirmation is taken, the notification processing function 158 is configured to display the message 281A “CONFIRMATION COMPLETED” in place of the message 281 “NEED TO CONFIRM”; however, possible embodiments are not limited to this example. For instance, the notification processing function 158 does not necessarily have to display a message when the action for a confirmation is taken. In other words, the display of the message 281 “NEED TO CONFIRM” may be ended.

When the action for an update is taken (step S301: Yes), the notification processing function 158 monitors, at step S304, whether or not an action on the link information is taken. The action on the link information may be, for example, the image interpreting doctor performing a click operation by placing a cursor on the link information 261 or 263 appended to the character string “9 mm” or “STAGE T4” in the observations in the observation display section 260, by using the input interface 110 (FIG. 2).

When an action on the link information is performed (step S304: Yes), the link processing function 157 links, at step S305, to the link destination (the updating means) indicated by the link information. For example, when the image interpreting doctor performs a click operation by placing a cursor on the link information 261 appended to the character string “9 mm” in the observations in the observation display section 260 while using the input interface 110, the link processing function 157 links to the link destination (the updating means) indicated by the link information 261. Further, the link processing function 157 receives an operation to update the update candidate item (the character string “9 mm”) to which the link information 261 is appended in the current medical report 200, by using the updating means linked by the link information 261.

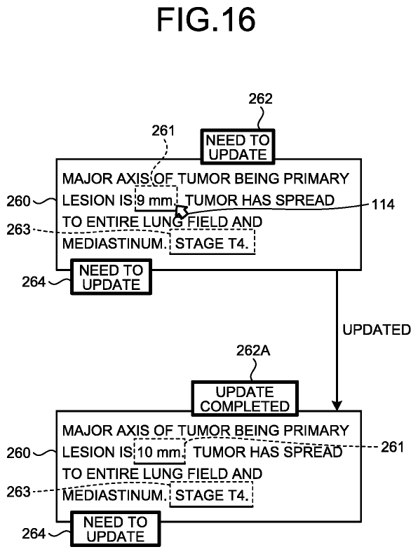

More specifically, as illustrated in FIG. 16, the image interpreting doctor performs the click operation by placing a cursor 114 on the link information 261 appended to the character string “9 mm” in the observations in the observation display section 260 while using the input interface 110. In that situation, the link processing function 157 links to the updating means “the image display viewer of Company A (the PACS server 40)” serving as the link destination indicated by the link information 261.

For example, by conducting a search while using the patient ID, the examination ID, the image ID, and the like, the link processing function 157 is able to obtain the medical image data generated during the examination performed at this time, from the PACS server 40. More specifically, as explained above, the medical image data saved in the PACS server 40 has appended thereto the additional information such as a patient ID, an examination ID, an image ID, and the like. Further, as explained above, the image ID contains, for example, the site of the examined subject (the patient) that was imaged, anatomical position information, the image generation time, the slice thickness, the slice position, and the like. Accordingly, by conducting a search while using the patient ID of the patient, the examination ID of the examination performed on the patient at this time, an image ID, and the like, the link processing function 157 is able to obtain, from the PACS server 40, such medical image data generated during the examination performed at this time (hereinafter, “medical image data acquired during the examination performed at this time”) that has the same anatomical position information as that of the image ID representing the primary lesion within the medical image data acquired during the past examination, from within the medical image data acquired during the examination performed at this time. More specifically, from among the medical image data generated during the past examination, the image interpreting doctor assigns the link information “9 mm” to such medical image data that represents the primary lesion. The image IDs from the past examination and the image IDs from the examination performed at this time have anatomical position information (e.g., an image ID=123 denotes an “S2 region of the liver”). Accordingly, the link processing function 157 is able to obtain, from the PACS server 40, such medical image data that has the same anatomical position information as that from the past examination, from among the medical image data acquired during the examination performed at this time. For instance, the medical image data obtained from the PACS server 40 is displayed on a display (the display 120 or the display 123). Accordingly, the link processing function 157 receives an operation to update the update candidate item (the character string “9 mm”) to which the link information 261 is appended in the current medical report 200, by using the updating means “the image display viewer of Company A (the PACS server 40)”.

Further, the image interpreting doctor measures the major axis of the tumor being the primary lesion, by using the input interface 110, with respect to the medical image data displayed on the display. As a result of the measuring process, let us assume that the major axis of the tumor being the primary lesion was “9 mm” in the past (immediately preceding) examination, but has changed to “10 mm” in the examination performed at this time. In that situation, as illustrated in FIG. 16, the image interpreting doctor performs an operation to update the character string “9 mm” in the observations in the observation display section 260 with a character string “10 mm”, by using the input interface 110. In other words, the character string “9 mm” in the observations in the observation display section 260 is updated with the character string “10 mm” in the current medical report 200.

When an action is taken on the link information (step S304: Yes) and after the link to the link destination is made (step S305), the notification processing function 158 checks to see whether or not an update has been performed at step S306.

When an update has been performed (step S306: Yes), the notification processing function 158 displays, at step S307, a message indicating that the character strings “9 mm” and “STAGE T4” of the update candidate items have been updated by the image interpreting doctor, in place of the messages 262 and 264 “NEED TO UPDATE”. For example, as illustrated in FIG. 16, the character string “9 mm” in the observations in the observation display section 260 is updated with the character string “10 mm”. In that situation, in place of the message 262 “NEED TO UPDATE”, the notification processing function 158 displays a message 262A “UPDATE COMPLETED” indicating the character string “9 mm” of the update candidate item has been updated by the image interpreting doctor. In other words, in the vicinity of the character string “10 mm” in the observations in the observation display section 260, the message 262A “UPDATE COMPLETED” is displayed in place of the message 262 “NEED TO UPDATE”. That is to say, the notification processing function 158 displays, in a recognizable manner in the observation display section 260, information indicating that the character string “9 mm” in the observation display section 260 has been updated with the character string “10 mm” by a manual update of the image interpreting doctor, as an update state of the update candidate item manually updated, by displaying the message 262A “UPDATE COMPLETED”.

When an action on the link information is taken, the notification processing function 158 is configured to display the message 262A “UPDATE COMPLETED” in place of the message 262 “NEED TO UPDATE”; however, possible embodiments are not limited to this example. For instance, the notification processing function 158 does not necessarily have to display a message when an action on the link information is taken. In other words, the display of the message 262 “NEED TO UPDATE” may be ended.

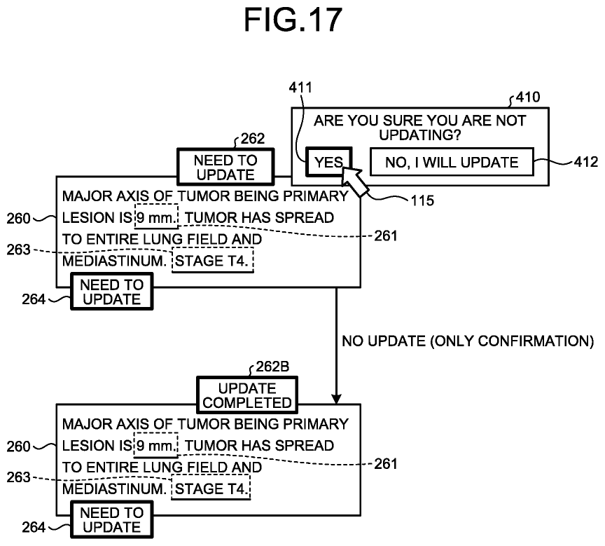

When no action on the link information is taken (step S304: No), but a click operation is performed by placing a cursor on the message 262 “NEED TO UPDATE” displayed in the vicinity of the character string “9 mm” in the observations in the observation display section 260, the notification processing function 158, at step S308, presents a confirmation display as illustrated in FIG. 17 for example. As another example, when an action on the link information is taken (step S304: Yes), the linking to the link destination is made (step S305), but no update is made (step S306: No), the notification processing function 158 presents a confirmation display.

In this situation, as illustrated in FIG. 17, the notification processing function 158 displays a message 410 “Are you sure you are not updating?” checking to see whether or not the update is unnecessary, with respect to the update candidate item (the character string “9 mm”) to which the link information 261 is appended. The message 410 “Are you sure you are not updating?” is displayed as being superimposed on the observation display section 260 like a pop-up screen. The message 410 contains a button 411 used for confirming that the update is unnecessary and another button 412 used for linking to the link destination indicated by the link information 261.

At step S309, the notification processing function 158 monitors operations performed on the buttons 411 and 412 in the message 410.

When the image interpreting doctor operates on the button 411 in the message 410 by using the input interface 110 (step S309: Yes), the notification processing function 158 displays, at step S310, a message indicating that the character strings “9 mm” and “STAGE T4” serving as the update candidate items have been confirmed by the image interpreting doctor, but will not be updated, in place of the messages 262 and 264 “NEED TO UPDATE”. For example, let us assume that, as a result of a measuring process, the major axis of the tumor being the primary lesion was “9 mm” in the past (immediately preceding) examination and was again “9 mm” in the examination performed at this time. In this situation, as illustrated in FIG. 17, the image interpreting doctor performs a click operation by placing a cursor 115 on the button 411 in the message 410 by using the input interface 110. In that situation, the notification processing function 158 displays a message 262B “CONFIRMATION COMPLETED” indicating that the character string “9 mm” serving as an update candidate item has been confirmed by the image interpreting doctor but will not be updated, in place of the message 262 “NEED TO UPDATE”. In other words, in the vicinity of the character string “9 mm” in the observations in the observation display section 260, the message 262B “CONFIRMATION COMPLETED” is displayed in place of the message 262 “NEED TO UPDATE”. That is to say, the notification processing function 158 displays, in a recognizable manner in the observation display section 260, information indicating that the image interpreting doctor who confirmed the character string “9 mm” in the observation display section 260 determines that the character string will not be updated, as an update state of the update candidate item to be manually updated, by displaying the message 262B “CONFIRMATION COMPLETED”.

In contrast, when the image interpreting doctor operates the button 412 in the message 410 by using the input interface 110 (step S309: No), the processes at step S304 and thereafter will be performed. In other words, the link processing function 157 links to the updating means “the image display viewer of Company A (the PACS server 40)” serving as the link destination indicated by the link information 261. Further, the link processing function 157 receives an operation to update, by using the updating means, the update candidate item (the character string “9 mm”) to which the link information 261 is appended in the current medical report 200.

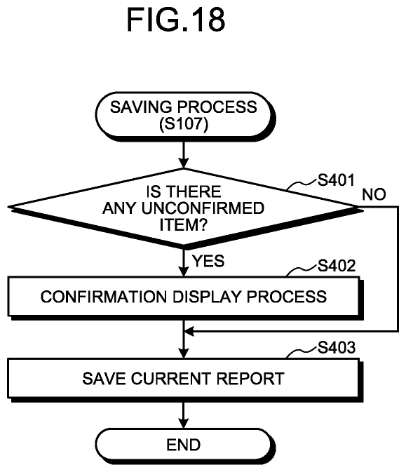

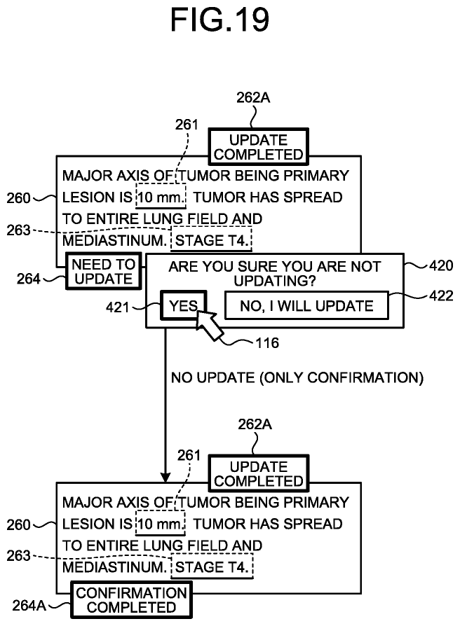

FIG. 18 is a flowchart illustrating a procedure in the saving process (step S107) according to the present embodiment. FIG. 19 is a drawing for explaining the saving process (step S107) according to the present embodiment.

Steps S401 and S403 in FIG. 18 are steps executed as a result of the processing circuitry 150 invoking a program corresponding to the saving processing function 159 of the report generating function 153 from the storage circuit 140. Step S402 in FIG. 18 is a step executed as a result of the processing circuitry 150 invoking a program corresponding to the notification processing function 158 and the link processing function 157 of the report generating function 153 from the storage circuit 140.

At step S401, when the image interpreting doctor issues a save instruction by using the input interface 110, the saving processing function 159 checks to see whether or not the template for the current medical report 200 has at least one unconfirmed character string or image.

When the template for the current medical report 200 has at least one unconfirmed character string or image (step S401: Yes), the notification processing function 158 and the link processing function 157 perform a confirmation display process at step S402. For example, in the update/confirmation process (step S106), the image interpreting doctor did not take an action for an update with respect to the character string “STAGE T4” in the observations in the observation display section 260. More specifically, the image interpreting doctor has not performed a click operation while a cursor is placed on either the link information 263 appended to the character string “STAGE T4” in the observations in the observation display section 260 or the message 264 “NEED TO UPDATE” displayed in the vicinity of the character string “STAGE T4”, by using the input interface 110.

In that situation, as illustrated in FIG. 19, the notification processing function 158 displays a message 420 “Are you sure you are not updating?” checking to see whether or not the update is unnecessary, with respect to the update candidate item (the character strong “STAGE T4”) to which the link information 263 is appended. The message 420 “Are you sure you are not updating?” is displayed as being superimposed on the observation display section 260 like a pop-up screen. The message 420 contains a button 421 used for confirming that the update is unnecessary and another button 422 used for linking to the link destination indicated by the link information 263.

For example, the image interpreting doctor performs a click operation while placing a cursor on the link information 263 appended to the character string “STAGE T4” in the observations in the observation display section 260 by using the input interface 110. In that situation, the link processing function 157 links to the updating means “the server of Company C (the medical dictionary server 60)” serving as the link destination indicated by the link information 263. Further, the link processing function 157 receives an operation to update, by using the updating means, the update candidate item (the character string “STAGE T4”) to which the link information 263 is appended in the current medical report 200. Accordingly, the image interpreting doctor reads the medical dictionary keeping measurement values and categories in correspondence with each other for each of various sites, from the medical dictionary server 60. The image interpreting doctor refers to the read medical dictionary and determines a stage of the cancer corresponding to the measurement value (the value of the major axis of the tumor being the primary lesion). Let us discuss an example in which, with reference to the dictionary, the stage of the cancer was “STAGE T4” in the past (immediately preceding) examination, and the stage is again “STAGE T4” in the examination performed at this time. In that situation, the image interpreting doctor does not update the character string “STAGE T4” in the observations in the observation display section 260.

Accordingly, as illustrated in FIG. 19, when the message 420 “Are you sure you are not updating?” is displayed in the observation display section 260, the image interpreting doctor performs a click operation while placing a cursor 116 on the button 421 in the message 420, by using the input interface 110. The notification processing function 158 displays a message 264A “CONFIRMATION COMPLETED” indicating that the character string “STAGE T4” serving as an update candidate item has been confirmed by the image interpreting doctor but will not be updated, in place of the message 264 “NEED TO UPDATE”. In other words, in the vicinity of the character string “STAGE T4” in the observations in the observation display section 260, the message 264A “CONFIRMATION COMPLETED” is displayed, in place of the message 264 “NEED TO UPDATE”. That is to say, the notification processing function 158 displays, in a recognizable manner in the observation display section 260, information indicating that the image interpreting doctor who confirmed the character string “STAGE T4” in the observation display section 260 determines that the character string will not be updated, as an update state of the update candidate item, by displaying the message 264A “CONFIRMATION COMPLETED”.

When the image interpreting doctor operates the button 422 in the message 420 by using the input interface 110, a link is made to the updating means “the server of Company C (the medical dictionary server 60)” serving as the link destination indicated by the link information 263. Further, the link processing function 157 receives an operation to update, by using the updating means, the update candidate item (the character string “STAGE T4”) to which the link information 263 is appended in the current medical report 200.

As explained above, at step S402, when there is an update candidate item that is not automatically updatable and has not been updated because the image interpreting doctor has not made the confirmation, the notification processing function 158 displays the message checking to see whether or not the update is unnecessary with respect to the update candidate item. In other words, with respect to the update candidate item to be manually updated, the notification processing function 158 is configured to prompt the doctor to confirm whether or not the update is unnecessary at the time of saving; however, possible embodiments are not limited to this example. The notification processing function 158 may prompt the doctor to confirm whether or not the confirmation is unnecessary with respect to an update candidate item to be automatically updated, at the time of saving.

For example, at step S402, when there is at least one update candidate item (the key image 280) that has automatically been updated and has not been confirmed, the notification processing function 158 displays a message (e.g., “Are you sure you are skipping the confirmation?”) checking to see whether or not the confirmation is unnecessary, with respect to the update candidate item (the key image 280). The message “Are you sure you are skipping the confirmation?” is displayed in the vicinity of the multimedia data display section 270 like a pup-up screen. The message contains a confirmation button to be operated when a confirmation is made. For example, the image interpreting doctor performs a click operation by placing a cursor on the confirmation button of the message, by using the input interface 110. In that situation, the notification processing function 158 displays the message 281A “CONFIRMATION COMPLETED” indicating that the confirmation of the key image 280 by the image interpreting doctor is completed, in place of the message 281 “NEED TO CONFIRM”. In other words, either on the inside or in the vicinity of the key image 280 in the multimedia data display section 270, the message 281A “CONFIRMATION COMPLETED” is displayed, in place of the message 281 “NEED TO CONFIRM”. That is to say, the notification processing function 158 displays, in a recognizable manner in the multimedia data display section 270, information indicating that the image interpreting doctor confirmed the key image 280 in the multimedia data display section 270, as an update state of the update candidate item automatically updated, by displaying the message 281A “CONFIRMATION COMPLETED”.

Further, in the above example, when the automatically-updated key image 280 was confirmed, the notification processing function 158 is configured to display the message 281A “CONFIRMATION COMPLETED” in place of the message 281 “NEED TO CONFIRM”; however, possible embodiments are not limited to this example. For instance, when the automatically-updated key image 280 is confirmed by the image interpreting doctor and is subsequently replaced with another key image selected by the image interpreting doctor, the notification processing function 158 may display a message “CONFIRMATION COMPLETED” in place of the message “NEED TO CONFIRM”. In other words, the notification processing function 158 may display, in a recognizable manner in the multimedia data display section 270, information indicating that the automatically-updated key image 280 was replaced with the key image selected by the image interpreting doctor, as an update state of the update candidate item automatically updated, by displaying the message “CONFIRMATION COMPLETED”.

When the image interpreting doctor issues the save instruction at step S403 by using the input interface 110, when the template for the current medical report 200 has no unconfirmed character string or image (step S401: No), the saving processing function 159 converts, the data format of the observations displayed in the observation display section 260 into text data (i.e., removes the link information 261 and 263) and generates the current medical report 200. In another example, when the image interpreting doctor issues a save instruction by using the input interface 110, when there is no unconfirmed character string or image because the process step S402 has been performed, the saving processing function 159 converts the data format of the observations displayed in the observation display section 260 into text data (removes the link information 261 and 263) and generates the current medical report 200. In this situation, the generated current medical report 200 is the medical report 200 to be saved and is an example of the second medical report.

The generated current medical report 200 is newly provided with a registered information display section containing the registration date “Oct. 11, 2018” on which the current medical report 200 is saved (registered) into the PACS server 40 and the name “XX” of the image interpreting doctor (the registerer) who generated the current medical report 200. Further, the saving processing function 159 saves (registers) the generated current medical report 200 into the PACS server 40. More specifically, the saving processing function 159 transits the generated current medical report 200 and the examination execution information corresponding to the current medical report 200 to the PACS server 40. In that situation, the PACS server 40 receives the current medical report 200 and the examination execution information corresponding to the current medical report 200 transmitted thereto from the medical report generating device 100 and further manages the current medical report 200 and the examination execution information that were received, so as to be kept in correspondence with each other.

As explained above, the medical report generating device 100 according to the present embodiment is configured to obtain the medical report generated in the past, to generate the template for the current medical report 200 on the basis of the past medical report 300, and to cause the display 123 to display, together with the template, the update candidate items (the measurement value “9 mm”, the category “STAGE T4”, and the key image) that need to be updated in the current medical report 200 in the recognizable manner. Thus, the medical report generating device 100 according to the present embodiment is configured to cause the display 123 to display the update candidate items that need to be updated in the current medical report 200, in the recognizable manner. As a result, the image interpreting doctor does not need to determine the update candidate items in the current medical report 200 for himself/herself. It is therefore possible to lessen the burden that might be imposed on the image interpreting doctor when he/she had to determine the update candidate items for himself/herself. In other words, the medical report generating device 100 according to the present embodiment is able to make the updating work efficient. Further, the medical report generating device 100 according to the present embodiment is able to prevent the image interpreting doctor from overlooking the update candidate items while generating the current medical report 200.

The constituent elements of the apparatuses and the devices described in the present embodiments are based on functional concepts. Thus, it is not necessary to physically configure the constituent elements as indicated in the drawings. In other words, specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program analyzed and executed by the CPU or may be realized as hardware using wired logic.

Further, it is possible to realize the methods described in the present embodiments by causing a computer such as a personal computer or a workstation to execute a program prepared in advance. It is possible to distribute the program via a network such as the Internet. Further, it is also possible to record the program onto a computer-readable non-transitory recording medium such as a hard disk, a flexible disk (FD), a Compact Disk Read-Only Memory (CD-ROM), a Magneto Optical (MO) disk, a Digital Versatile Disk (DVD), or the like, so as to be executed as being read from the recording medium by a computer.

As explained above, according to at least one aspect of the present embodiments, it is possible to lessen the burden related to the generation of the medical reports.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical report generating device comprising processing circuitry configured:
to obtain a medical report generated in a past;
to generate a first medical report serving as a current medical report on a basis of the past medical report and to identify at least one update candidate item that needs to be updated in the first medical report; and
to cause a display to display the identified update candidate item in a recognizable manner.

2. The medical report generating device according to claim 1, wherein the processing circuitry causes a display to display an update state of the identified update candidate item in a recognizable manner.

3. The medical report generating device according to claim 2, wherein
the processing circuitry determines whether or not the identified update candidate item is automatically updatable in the first medical report, and
in accordance with whether or not the update candidate item is automatically updatable, the processing circuitry causes the display to display the update state of the identified update candidate item in the recognizable manner.

4. The medical report generating device according to claim 3, wherein, by referring to information keeping, in correspondence with one another, a type of the update candidate item, an updating method for updating the update candidate item, and automatic update capability information indicating whether or not the updating method is automatically updatable, the processing circuitry determines whether or not the identified update candidate item is automatically updatable in the first medical report.

5. The medical report generating device according to claim 3, wherein, when having determined that the identified update candidate item is automatically updatable, the processing circuitry automatically updates the identified update candidate item in the first medical report, by using an updating method corresponding to the identified update candidate item.

6. The medical report generating device according to claim 5, wherein, among the identified update candidate items, the processing circuitry causes the display to display, in a recognizable manner, one or more already-updated items which are update candidate items that were automatically updated; and one or more update requiring items which are update candidate items that are not automatically updatable and which require an update.

7. The medical report generating device according to claim 6, wherein, among the identified update candidate items, the processing circuitry displays a message indicating that an update was performed with respect to one or more update candidate items that were automatically updated and displays a message indicating that an update is necessary with respect to one or more update candidate items that are not automatically updatable.

8. The medical report generating device according to claim 7, wherein the processing circuitry displays a message indicating that a confirmation is necessary, as the message indicating that the update was performed with respect to the one or more update candidate items that were automatically updated.

9. The medical report generating device according to claim 3, wherein, when the first medical report is to be saved as a second medical report, the processing circuitry displays a message checking to see whether or not an update is unnecessary with respect to the update candidate item that is not automatically updatable and has not been updated.

10. The medical report generating device according to claim 3, wherein, when the first medical report is to be saved as a second medical report, the processing circuitry displays a message checking to see whether or not a confirmation is unnecessary with respect to the update candidate item that has automatically been updated and has not been confirmed.

11. The medical report generating device according to claim 3, wherein, when having determined that the identified update candidate item is not automatically updatable, the processing circuitry appends link information linking to an updating method corresponding to the identified update candidate item as a link destination, to the identified update candidate item in the first medical report.

12. The medical report generating device according to claim 11, wherein the processing circuitry displays a message indicating that an update is necessary with respect to the update candidate item to which the link information is appended.

13. The medical report generating device according to claim 11, wherein, by using the updating method linked by the link information, the processing circuitry receives an operation to update the update candidate item to which the link information is appended in the first medical report.

14. The medical report generating device according to claim 11, wherein, when the update candidate item to which the link information is appended in the first medical report was not updated, the processing circuitry displays a message checking to see whether or not an update is unnecessary with respect to the update candidate item to which the link information is appended.

15. The medical report generating device according to claim 11, wherein when no link is made by the link information at a time of saving the first medical report as a second medical report, the processing circuitry displays a message checking to see whether or not an update is unnecessary with respect to the update candidate item to which the link information is appended.

16. A medical report generating method comprising:
obtaining a medical report generated in a past;
generating a first medical report serving as a current medical report on a basis of the past medical report and identifying at least one update candidate item that needs to be updated in the first medical report; and
causing a display to display the identified update candidate item in a recognizable manner.
